(19) United States
(12) Patent Application Publication
(10) Pub. No.: US 2007/0265287 A1
(43) Pub. Date: Nov. 15, 2007

(54) Method for Producing 2-Amino-4,6-Dichloro-5-Formamidopyrimidine

(75) Inventors: Thomas Guthner, Trostberg (DE); Karl-Heinz Neuhauser, Trostberg (DE)

Correspondence Address: LAW OFFICE OF MICHAEL A. SANZO, LLC, 15400 CALHOUN DR., SUITE 125, ROCKVILLE, MD, 20855, US

(73) Assignee: DEGISSA AG, DUSSELDORF (DE)

(21) Appl. No.: 10/585,727

(22) PCT Filed: Jan. 14, 2005
(86) PCT No.: PCT/EP05/00325

§ 371 Date: Mar. 17, 2007

(30) Foreign Application Priority Data
Jan. 15, 2004 (DE) 10 2004 002 055.8

(51) Int. Cl. A61K 31/505; A61P 31/12; C07D 239/30

(52) U.S. Cl. 514/275; 544/297

(57) ABSTRACT

The invention relates to a method for producing 2-amino-4,6-dichloro-5-formamidopyrimidine from 2,5-diamino-4,6-dihydroxypyrimidine or a salt thereof. According to said method, a) 2,5-diamino-4,6-dihydroxypyrimidine or the salt or tautomer forms thereof are reacted with a chlorination agent and a formamide of formula (I) wherein R¹ and R² independently represent a C₁-C₄ alkyl radical or —R¹-R²— represents —(CH2)ₙ— where n=4-6, or —(CH₂)₂-0-(CH₂)₂—, without adding a solvent, at a temperature of between 50 and 130° C., b) the reaction product obtained in step a) is reacted with water at a temperature of between 0 and 100° C., and regulated to a pH value of between 1 and 6 with an inorganic base, and c) the aqueous reaction mixture obtained in step b) is left to react at a temperature of between 70 and 120° C. under hydrolysis to form 2-amino-4,6-dichloor-5-formamidopyrimidine. The inventive method enables satisfactory yields and high levels of purity of the end product to be obtained. As a result of the significantly reduced reaction volumes due to the solvent, auxiliary substances and residual substances saved, and the significantly simplified method, the costs incurred in the production of 2-amino-4,6-dichloro-5-formylaminopyrimidine are significantly reduced.

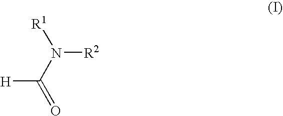

2-Amino-4,6-dichloro-5-formamidopyrimidine is a valuable intermediate for the preparation of purine derivatives, as find use, for example, as active pharmaceutical ingredients in the treatment of viral diseases, for example in the treatment of AIDS. Such a medicament and routes to its preparation have been described in detail (cf. Susan M. Daluge et. al., Nucleosides, Nucleotides & Nucleic Acids, 19(1&2), 297-327 (2000)).

Routes for the synthesis of 2-amino-4,6-dichloro-5-formamidopyrimidine have already been described in accordance with the prior art. The known processes are based on the principle that 2,5-diamino-4,6-dihydroxypyrimidine (or a salt thereof) is reacted with a chlorinating agent and a formamide and/or a Vilsmeyer-type reagent. In this process, the amino groups, which tend to side reactions in unprotected form, are protected as formamidines, the hydroxyl groups are chlorinated and the protecting groups are removed again partially of fully in subsequent steps. The overall synthesis sequence can be illustrated with the following scheme:

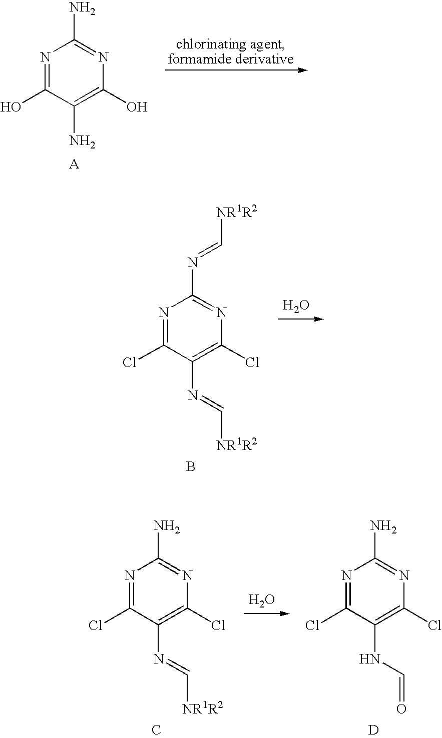

This reaction has already been considered in detail in the patent literature. For example, U.S. Pat. No. 6,552,193 describes the reaction of 2,5-diamino-4,6-dihydroxypyrimidine hemisulfate A with chloromethylenedimethylammonium chloride (Vilsmeyer reagent) in chloroform to give B (R¹, R²═CH₃) in 81% yield, the hydrolysis thereof to give C in 95% yield and the further reaction of C to give 2-amino-4,6-dichloro-5-formamidopyrimidine D in a phosphate buffer with 68% yield. The overall yield over all 3 stages is 52%. According to the teaching from U.S. Pat. No. 6,552,193, an inert solvent, for example dichloromethane, chloroform or dichloroethane, is required for the first reaction step (the chlorination).

U.S. Pat. No. 5,663,340 and EP 684 236 describe the chlorination of A with phosphorus oxychloride in the presence of dimethylformamide using an inert solvent (examples include toluene, xylene, chloroform, dichloromethane, dichloroethane, chlorobenzene) to form B (R¹, R²═CH₃) and further reaction to give C (without isolation of B) in 85% yield. The conversion of C to D is effected in the presence of aqueous propionic acid in 64% yield. The overall yield is thus 54%.

Although these prior art processes mentioned fulfill their purpose of providing 2-amino-4,6-dichloro-5-formamidopyrimidine D as an intermediate for preparing antiviral pharmaceuticals, they display significant disadvantages. For the chlorination step a), considerable amounts of chlorinated and/or aromatic solvents are used. This gives rise to an unfavorable space-time yield and considerable environmental pollution. The salt-containing wastewaters obtained from the chlorination step have to be disposed of, but a reagent (phosphate buffer or propionic acid) again has to be used for the subsequent hydrolysis step from C to D.

In addition, it is not evident from the prior art—in spite of some experiments with “one-pot variants”—that a direct process for preparing D from A without isolating the intermediates might be possible. All of these factors increase the amount of the raw materials used and the residual materials to be disposed of, worsen the space-time yield in the production and lead additionally to considerable environmental pollution.

It was therefore an object of the invention to provide a simple and environmentally friendly process for preparing 2-amino-4,6-dichloro-5-formamidopyrimidine from 2,5-diamino-4,6-dihydroxypyrimidine or a salt thereof with high space-time yields. In addition, the process should be simplified such that the isolation of intermediates can be dispensed with (one-pot reaction).

The object of the invention is achieved by
a) reacting the 2,5-diamino-4,6-dihydroxypyrimidine or salt or tautomeric forms thereof with a chlorinating agent and a formamide of the formula (I) 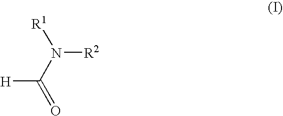
where
R¹ and R² are each independently a C₁-C₄-alkyl radical, or —R¹ -R²— is —(CH₂)ₙ— where n=from 4 to 6 or —(CH₂)₂—O—(CH₂)₂—, without addition of a solvent at from 50 to 130° C.,
b) reacting the reaction product from stage a) at from 0 to 100° C. with water and adjusting to a pH of from 1.0 to 6.0 with an inorganic base and
c) reacting the aqueous reaction mixture from stage b) at from 70 to 120° C. with hydrolysis to give 2-amino-4,6-dichloro-5-formamidopyrimidine.

Surprisingly, it has been found that, in a suitable reaction, the reaction mixture in the chlorination step is better stirrable without solvent than when a solvent is used, and that, owing to the solubility conditions of product and by-products, 2-amino-4,6-dichloro-5-formamidopyrimidine can be obtained in high purity from the complex reaction mixture.

The raw material used for the process according to the invention is 2,5-diamino-4,6-dihydroxypyrimidine or salt or tautomeric forms thereof. Owing to the easy oxidizability of the free base, especially the hemisulfate, the hydrochloride monohydrate and the anhydrous hydrochloride are particularly suitable. In order to avoid unnecessary reagent consumption and undesired sulfate ions, particular preference is given to using anhydrous 2,5-diamino-4,6-dihydroxypyrimidine hydrochloride.

The chlorinating agents used may be various inorganic and organic reagents having the functionality of an acid chloride. Examples include phosgene, oxalyl chloride, chloromethylenedimethylammonium chloride (Vilsmeyer reagent), thionyl chloride, sulfuryl chloride, phosphorus trichloride, phosphorus pentachloride or phosphorus oxychloride. Particular preference is given to using phosphorus oxychloride.

The formamide of the formula (I) serves to formylate the amino groups of the starting material and to protect them as the formamidine.

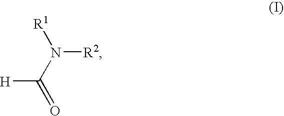

The intermediates obtained in stage a) are the 2,5-diformamidino-4,6-dichloropyrimidines of the formula (II):

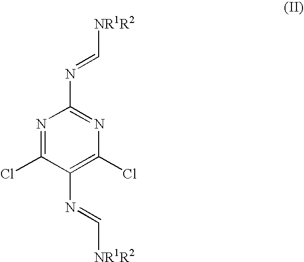

The particular R¹ and R² radicals are detached again in the further reaction to give the end product, so that, irrespective of the amide of the formula I used, the same end product is always obtained. The R¹ and R² radicals are each independently a C₁ to C₄-alkyl radical and in particular methyl, ethyl, n-propyl or/and n-butyl. Alternatively, the R¹ and R² radicals can be joined via a single bond and can assume the definition —(CH₂)ₙ— where n=from 4 to 6 or —(CH₂)₂—O—(CH₂)₂—. Preferred amides of the formula (I) are N,N-dimethylformamide, N-formylpyrrolidine, N-formylpiperidine and N-formylmorpholine. Particular preference is given to N,N-dimethylformamide.

The molar ratios of the reactants in the chlorination step can be varied within wide limits. Preference is given to using from 1 to 5 mol of formamide of the formula (I) per 1 mol of 2,5-diamino-4,6-dihydroxypyrimidine. Preference is further given to using from 3 to 7 mol of chlorinating agent per 1 mol of 2,5-diamino-4,6-dihydroxypyrimidine. For the special case of use of phosphorus oxychloride and N,N-dimethylformamide, preference is given to using from 3 to 5 mol of phosphorus oxychloride and from 1 to 3 mol of N,N-dimethylformamide per mole of 2,5-diamino-4,6-dihydroxypyrimidine.

In a preferred embodiment, the chlorinating agent is first mixed with the formamide and only in a second step is the 2,5-diamino-4,6-dihydroxypyrimidine, as the case may be, metered in slowly or added in portions. In this way, it is ensured that the insoluble 2,5-diamino-4,6-dihydroxypyrimidine added is reacted continually to give soluble subsequent products of the structure (II), so that stirrability remains ensured.

In a preferred embodiment, the chlorinating agent is initially charged. The N,N-dialkylformamide is then added at a temperature of from 20 to 100° C., preferably from 40 to 70° C., and the reaction mixture is allowed to react at this temperature for a period of from 5 to 180 minutes. The 2,5-diamino-4,6-dihydroxypyrimidine is metered in at a temperature of from 50 to 130° C., preferably from 50 to 100° C., over the course of from 15 minutes to 5 hours. Subsequently, continued reaction is effected over from 1 to 30 hours at a temperature of from 50 to 130° C., preferably from 70 to 110° C.

In a preferred embodiment, reaction step a) is effected within a temperature range from 70 to 110° C.

The subsequent hydrolysis step can in principle be carried out in two different ways. One is to meter the amount of water required directly into the chlorination mixture. This is advantageous since no further reaction vessel is required, but has the disadvantage of a metering time which is longer owing to the high heat production. Alternatively, with the same result, the chlorination mixture can be metered into initially charged water.

The added or initially charged water should be sufficient, after the end of the hydrolysis, to obtain a readily stirrable reaction mixture. According to the invention, from 2 to 5 liters of water per 1 mol of 2,5-diamino-4,6-dihydroxypyrimidine used are sufficient for this purpose.

The hydrolysis step b) should be effected within the temperature range from 0 to 100° C. The range from 20 to 60° C. is considered to be preferable.

Subsequently, the resulting reaction mixture is adjusted to a certain pH with an inorganic base and partially hydrolyzed in this way.

Suitable inorganic bases are in principle all bases which form soluble chloride salts. Preference is given to sodium hydroxide solution, sodium hydroxide, sodium carbonate, sodium hydrogencarbonate, potassium hydroxide solution, potassium hydroxide, potassium carbonate and potassium hydrogencarbonate. Particular preference is given to sodium hydroxide solution. The amount of base added depends upon the pH to be established and it is typically from 2 to 3 mol per mole of chlorinating agent used.

The pH is crucial, since it controls the selective reaction of B via C to D. In the case of incorrectly selected pH, a reduced yield and/or undesired by-products in the product are obtained. According to the invention, the pH is adjusted at a defined value in the range between pH 1.0 and 6.0, preferably from pH 2.0 to 5.0, more preferably from 3.0 to 4.0, the pH being measured by means of a glass electrode at a temperature of 20° C. If appropriate, the pH can be readjusted continuously in the course of the reaction which follows by adding further base under pH control.

The further reaction is carried out by heating the aqueous mixture to a temperature of from 70 to 120° C., preferably from 80 to 100° C. In the course of a reaction time of from 1 to 20 hours, the unisolated intermediates form the desired target product 2-amino-4,6-dichloro-5-formamidopyrimidine. This is insoluble in the reaction mixture and can be removed, washed and dried by means of process steps familiar to those skilled in the art.

It is considered to be essential to the invention that this last reaction step is effected in the absence—even of traces—of a solvent. This is because it has been found to be capable of starting to dissolve the water-insoluble 2-amino-4,6-dichloro-5-formamidopyrimidine in the reaction mixture, which makes the pyrimidine more vulnerable to a further hydrolysis, so that the ultimate result is reduced yields and/or contamination of the product with 2,5-diamino-4,6-dichloropyrimidine, the subsequent product of the hydrolysis.

The process according to the invention affords satisfactory yields which are only slightly below the yields of the prior art processes. On the other hand, it offers the advantage of higher purity of the end products. The considerably reduced reaction volumes, the savings of solvents, assistants and residual substances, and the considerably simplified process in process technology terms, give rise to distinctly more favorable preparation costs for 2-amino-4,6-dichloro-5-formylaminopyrimidine.

A further aspect of the invention relates to the use of the 2-amino-4,6-dichloro-5-formylaminopyrimidine prepared by the process according to the invention for preparing purine derivatives. The invention further relates to the use of the 2-amino-4,6-dichloro-5-formylaminopyrimidine prepared by the process according to the invention for preparing active pharmaceutical ingredients, in particular for antiviral medicaments, for example for the treatment of AIDS.

The examples which follow serve to illustrate the process found without restricting the scope of the invention.

### EXAMPLES

### Example 1

61.33 g (0.40 mol) of phosphorus oxychloride were initially charged. At 50° C., 18.27 g (0.25 mol) of dimethylformamide were added dropwise within 45 minutes. The mixture was then heated to 70° C. and 17.86 g (0.10 mol) of 2,5-diamino-4,6-dihydroxypyrimidine hydrochloride were added by spatula within 45 minutes. Subsequently, the mixture was heated to 90° C. and stirred for 20 hours. A dark, moderately viscous, but homogeneous and readily stirrable mixture formed. It was cooled to 20° C. and admixed with 200 g of water with external cooling. Addition of 82.03 g of 50% sodium hydroxide solution adjusted the pH from −0.6 to 4.0, and the reaction mixture was heated to 90° C. and stirred for 8 hours. The mixture was cooled to 18° C., and the precipitated product was filtered off with suction, washed with water and dried under reduced pressure.

8.28 g of pure 2-amino-4,6-dichloro-5-formamidopyrimidine were obtained with a content of 98.7%. The yield based on 2,5-diamino-4,6-dihydroxypyrimidine used was 39.5%.

### Example 2

61.33 g (0.40 mol) of phosphorus oxychloride were initially charged and heated to 50° C. Within 45 minutes, 29.24 g (0.40 mol) of dimethylformamide were added dropwise. The mixture was then heated to 72° C. and 17.86 g (0.10 mol) of 2,5-diamino-4,6-dihydroxypyrimidine hydrochloride were added within 45 minutes. The mixture was heated to 90° C. and stirred for 17 hours. The mixture was then cooled to 20° C. and admixed with 200 g of water with external cooling. Addition of 88.35 g of 50% sodium hydroxide solution adjusted the pH from −0.5 to 3.6, and the reaction mixture was heated to 97° C. and stirred for 4 hours. The mixture was cooled to 18° C., and the precipitated product was filtered off with suction, washed with water and dried under reduced pressure.

7.92 g of pure 2-amino-4,6-dichloro-5-formamidopyrimidine were obtained with a content of 97.6%. The yield based on 2,5-diamino-4,6-dihydroxypyrimidine used was 37.4%.

### Example 3

### Comparative

180 ml of toluene and 76.7 g (0.5 mol) of phosphorus oxychloride were initially charged. At 50° C., 29.2 g of dimethylformamide were added dropwise within 45 minutes. At 70° C., 17.86 g (0.1 mol) of 2,5-diamino-4,6-dihydroxypyrimidine hydrochloride were then added in portions. Subsequently, the mixture was stirred at 90° C. for 20 hours. A viscous mass formed, which adhered to stirrer and flask wall and was only partially soluble in toluene.

After cooling, 300 g of water were metered into the mixture, the pH was adjusted to 5.0 by adding 89.6 g of 50% sodium hydroxide solution and the toluene phase was removed. An interface layer which was difficult to remove formed. After the toluene phase had been evaporated, 15.9 g of crude 2,5-bis(dimethylaminomethyleneamino)-4,6-dichloropyrimidine remained.

The water phase was extracted 3 times with 200 ml each time of ethyl acetate, and the organic phases were concentrated by evaporation. 9.8 g of a second, less pure fraction of 2,5-bis(dimethylaminomethyleneamino)-4,6-dichloropyrimidine remained.

250 g of water and 5.7 g of 85% phosphoric acid were initially charged, and adjusted to pH 4.0 with 3.7 g of sodium hydroxide solution, and the mixture of the two crude products was added. The mixture was again adjusted to pH 4.0 with 10.5 g of phosphoric acid. The mixture was stirred at 100° C. for 4 hours. After cooling, the precipitated product was filtered off, washed and dried. 11.3 g of 2-amino-4,6-dichloro-5-formamidopyrimidine were obtained with a content of 83.8%. The pure yield based on 2,5-diamino-4,6-dihydroxypyrimidine used was 45.7%.

An experiment carried out analogously using chlorobenzene instead of toluene lead to better stirrability of the reaction mixture. After extracting 3 times with chlorobenzene and analogous reaction in a phosphate buffer, 12.4 g of 2-amino-4,6-dichloro-5-formamidopyrimidine were obtained with a content of 78.3%. It was found that chlorobenzene distilled off incompletely had brought about partial further hydrolysis to 2,5-diamino-4,6-dichloropyrimidine.

1-20. (canceled)

21. A process for preparing 2-amino-4,6-dichloro-5-formamidopyrimidine from 2,5-diamino-4,6-dihydroxypyrimidine, or a salt or tautomeric form thereof, comprising:
a) reacting said 2,5-diamino-4,6-dihydroxypyrimidine, salt or tautomeric form with a chlorinating agent and a formamide of formula (I) 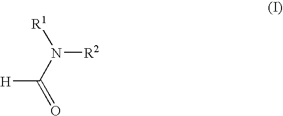
Wherein
R¹ and R² are each independently: a C₁-C₄-alkyl radical; or are joined together to form the ring —(CH₂)ₙ— where n is an integer from 4 to 6; or together form the ring —(CH₂)₂—O—(CH₂)₂—;
wherein the reaction is carried out without the addition of a solvent and at a temperature of from 50 to 130° C.;
b) reacting the product produced in the reaction of step a) with water at a temperature of from 0 to 100° C. and then adjusting the pH to between 1.0 and 6.0 with an inorganic base; and
c) hydrolyzing the aqueous reaction mixture produced in step b) at a temperature from 70 to 120° C. to give 2-amino-4,6-dichloro-5-formamidopyrimidine.

22. The process of claim 21, wherein the starting material used is 2,5-diamino-4,6-dihydroxypyrimidine in the form of a hemisulfate, hydrochloride monohydrate or as an anhydrous hydrochloride.

23. The process of claim 21, wherein the starting material used is anhydrous 2,5-diamino-4,6-dihydroxypyrimidine hydrochloride.

24. The process of claim 21, wherein said chlorinating agent is an acid chloride.

25. The process of claim 24, wherein said chlorinating agent is selected from the group consisting of phosgene; oxalyl chloride; chloromethylenedimethylammonium chloride; thionyl chloride; sulfuryl chloride; phosphorus trichloride; phosphorus pentachloride; and phosphorus oxychloride.

26. The process of claim 21, wherein the formamide of formula (I) is first reacted with said chlorinating agent and 2,5-diamino-4,6-dihydroxypyrimidine is then added.

27. The process of claim 21, wherein the formamide of formula I is selected from the group consisting of: N,N-dimethylformamide; N-formylpyrrolidine; N-formylpiperidine; N-formylmorpholine; and N,N-dimethylformamide.

28. The process of claim 21, wherein from 1.0 to 5.0 mol of formamide of formula (I) are used per mole of 2,5-diamino-4,6-dihydroxypyrimidine.

29. The process of claim 28 wherein from 3.0 to 7.0 mol of chlorinating agent are used per mole of 2,5-diamino-4,6-dihydroxypyrimidine.

30. The process of claim 21, wherein the reaction step a) is carried out within a temperature range of from 70 to 110° C.

31. The process of claim 21, wherein the inorganic base used in step b) is a base which forms soluble chloride salts.

32. The process of claim 21, wherein the inorganic base used in step b) is selected from the group consisting of: sodium hydroxide solution; sodium hydroxide; sodium carbonate; sodium hydrogencarbonate; potassium hydroxide solution; potassium hydroxide; potassium carbonate; and potassium hydrogencarbonate.

33. The process of claim 32, wherein the inorganic base used in step b) is sodium hydroxide solution.

34. The process of claim 21, wherein from 2 to 3 mol of inorganic base are used per mole of chlorinating agent.

35. The process of claim 21, wherein, in the neutralization in step b), pH is adjusted to between 2.0 and 5.0.

36. The process of claim 35, wherein, in the neutralization in step b), pH is adjusted to between 3.0 and 4.0.

37. The process of claim 36, wherein the reaction product from step a) is reacted at a temperature of from 20 to 60° C.

38. The process of claim 37, wherein the hydrolysis in step c) is carried out at a temperature of 70-120° C.

39. The process of claim 37, wherein the hydrolysis in step c) is carried out at a temperature of 80 to 100° C.

40. The process of claim 21, wherein step c) is carried out in the absence of an added solvent.

41. The process of claim 21, wherein said process is carried out without the isolation of intermediates, as a one-pot reaction.

42. A process for preparing purine derivatives, comprising the process steps of claim 20, and further comprising the conversion of 2-amino-4,6-dichloro-5-formamidopyrimidine to a purine derivative.

43. A process for preparing an active pharmaceutical ingredient, comprising the process steps of claim 40 and further comprising the conversion of said purine derivative to said active pharmaceutical ingredient.

44. The process of claim 43, wherein said active pharmaceutical ingredient is an antiviral medicament.

45. The process of claim 44, wherein said antiviral medicament is a medicament for the treatment of AIDS.
